(19) United States
(12) Patent Application Publication
(10) Pub. No.: US 2023/0197702 A1
(43) Pub. Date: Jun. 22, 2023

(54) SUNK-TYPE PACKAGE STRUCTURE

(71) Applicant: EMINENT ELECTRONIC TECHNOLOGY CORP. LTD., Hsin-Chu (TW)

(72) Inventors: Zhong-Hao DENG, New Taipei City (TW); Chien-Yu Huang, Taichung City (TW); Yi-Yung CHEN, New Taipei City (TW); KAO-PIN WU, New Taipei City (TW)

(21) Appl. No.: 17/990,158

(22) Filed: Nov. 18, 2022

Related U.S. Application Data
(60) Provisional application No. 63/290,655, filed on Dec. 17, 2021.

(30) Foreign Application Priority Data
Aug. 18, 2022 (TW) 111131124

(51) Int. Cl. H01L 25/16; H01L 23/053; H01L 23/552; H01L 31/0232

(52) U.S. Cl. CPC H01L 25/167; H01L 25/165; H01L 23/053; H01L 23/552; H01L 31/02327; H01L 2224/48227; H01L 24/48

(57) ABSTRACT

A sunk-type package structure includes a substrate, an optical sensing chip, and a housing. The substrate has a cavity with a first depth. The optical sensing chip is disposed inside the cavity and electrically connected with the substrate. A surface of the optical sensing chip has a first sensing area for sensing light. The housing covers the substrate and the optical sensing chip. The housing has a light-permeable portion disposed above the first sensing area.

This application claims priority of Application No. 111131124 filed in Taiwan on 18 Aug. 2022 under 35 U.S.C. § 119; and this application claims priority of U.S. Provisional Application No. 63/290,655 filed on 17 Dec. 2021 under 35 U.S.C. § 119(e); the entire contents of all of which are hereby incorporated by reference.

### BACKGROUND OF THE INVENTION

### Field of the Invention

The present invention relates to a package structure of an optical sensor, particularly to a sunk-type package structure of an optical sensor.

### Description of the Prior Art

Optical sensors are used in wearable devices or mobile devices to sense light. According to the sensing results of the optical sensor, the wearable device or mobile device can determine the ambient light intensity, the distance to an object, or the heartbeat rate of a user. Because the interior space of a wearable or mobile device (such as a true wireless stereo (TWS) or a smart watch) is very limited, the manufacturers pay much attention to the sizes of optical sensor packages.

FIG. 1 shows a conventional package structure 10 of an optical sensor. The package structure 10 comprises a substrate 10, a light source chip 12, an optical sensing chip 15, and a housing 18. The light source chip 12 and the optical sensing chip 15 are fixed to the substrate 11 with adhesives 13 and 16. Wires 14 and 17 respectively electrically connect the light source chip 12 and the optical sensing chip 15 with conduction pads 111 and 112 of the substrate 11. The housing 18 is disposed on the substrate 11, accommodating and covering the light source chip 12 and the optical sensing chip 15.

The conventional methods to reduce the size of the package structure 10 include: (1) optimizing the design of the optical sensing chip 15 to reduce the chip area, (2) thinning the light source chip 12 to decrease the chip thickness, and (3) promoting the process capability to lessen the package tolerance. However, optimizing the design of the optical sensing chip 15 would consume a lot of time and manpower or needs to adopt a further advanced fabrication process. In other words, the manufacturers have to pay more cost for optimizing the design of the optical sensing chip 15. Thinning the light source chip 12 may degrade performance and increase difficulties of manufacturers. Lessening the package tolerance means increasing fabrication risk and varying cost for the manufacturers. Besides, the existing fabrication process is not suitable to reduce the package tolerance any more.

As mentioned above, the existing methods to reduce the size of the package structure 10 of the optical sensor require the cooperation of all the suppliers, such that the methods are hard to practice and need much higher cost.

### SUMMARY OF THE INVENTION

One objective of the present invention is to provide a sunk-type package structure for an optical sensor.

According to one embodiment, the sunk-type package structure of an optical sensor comprises a substrate, an optical sensing chip, and a housing. The substrate has a cavity with a first depth. The optical sensing chip is disposed inside the cavity and electrically connected with the substrate. The surface of the optical sensing chip has a first sensing area for sensing light. The housing covers the substrate and the optical sensing chip. The housing includes a light-permeable area positioned above the first sensing area.

By disposing the optical sensing chip in the cavity of the substrate, the present invention can decrease the size of the optical sensor.

### BRIEF DESCRIPTION OF THE DRAWINGS

FIG. 1 shows a conventional package structure of an optical sensor.

FIG. 2 schematically shows a first embodiment of a proximity sensor applying the sunk-type package structure of the present invention.

FIG. 3 schematically shows another embodiment of the housing in FIG. 2.

FIG. 4 schematically shows a second embodiment of a proximity sensor applying the sunk-type package structure of the present invention.

FIG. 5 schematically shows a first embodiment of an ambient light sensor applying the sunk-type package structure of the present invention.

FIG. 6 schematically shows a second embodiment of an ambient light sensor applying the sunk-type package structure of the present invention.

FIG. 7 schematically shows an embodiment of a heartbeat sensor applying the sunk-type package structure of the present invention.

### DETAILED DESCRIPTION OF THE INVENTION

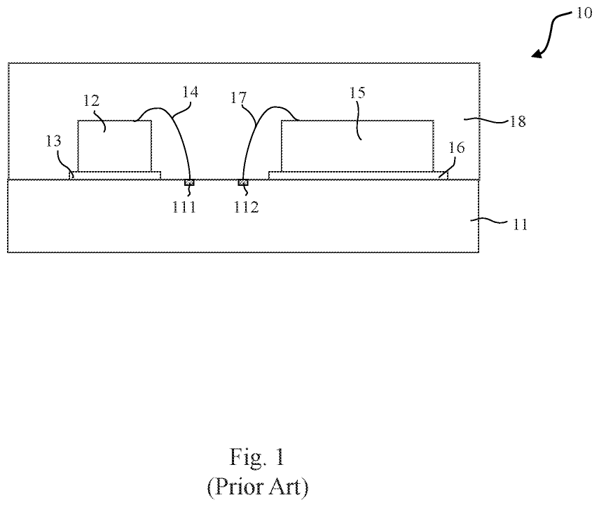

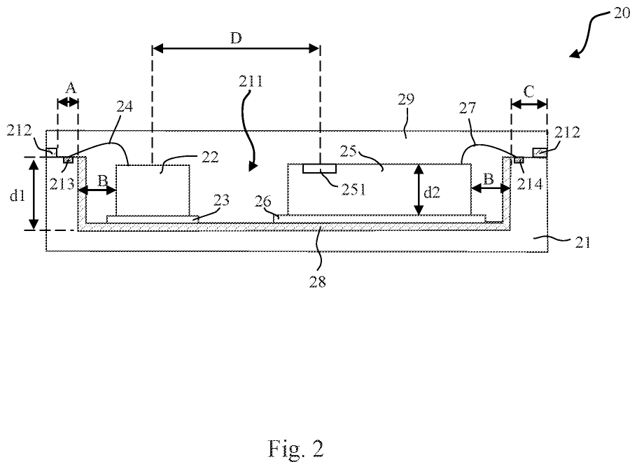

FIG. 2 schematically shows a first embodiment of a proximity sensor applying the sunk-type package structure of the present invention. The proximity sensor 20 shown in FIG. 2 comprises a substrate 21, a light source chip 22, an optical sensing chip 25, a metal film 28, and a housing 29. The substrate 21 has a cavity 211 with a first depth d1. The metal film 28 is formed on the surfaces of the inner walls and bottom of the cavity 211. The metal film 28 is used to reduce the electromagnetic interference (EMI) from the environment and the optical crosstalk interference from the substrate 21, whereby to enhance the sensing performance of the optical sensing chip 25. The light source chip 22 and the optical sensing chip 25 are disposed inside the cavity 211. The light source chip 22 is fixed to the metal film 28 on the bottom of the cavity 211 with an adhesive 23 and electrically connected with a conduction pad 213 of the substrate 21 through a wire 24. The light source chip 22 may be but is not limited to a LED chip or a Vertical-Cavity Surface-Emitting Laser (VCSEL) chip. The optical sensing chip 25 is fixed to the metal film 28 on the bottom of the cavity 211 with an adhesive 26 and electrically connected with a conduction pad 214 of the substrate 21 through a wire 27. The surface of the optical sensing chip 25 has a first sensing area 251 for sensing light. The housing 29 covers the substrate 21, the light source chip 22, and the optical sensing chip 25. In the embodiment shown in FIG. 2, the housing 29 is made of a fully-transparent material. In other words, the complete housing 29 is a light-permeable portion allowing light to pass. In another embodiment, the housing 29 is made of an opaque material; a hole is formed on a region of the housing 29, which is above the light source chip 22 and the first sensing area 251, to function as a light-permeable portion allowing light to pass. In one embodiment, the light source chip 22 is a VCSEL chip; the light emitted by the light source chip 22 is transmitted to the exterior of the proximity sensor 20 through the hole of the housing 29 (i.e., the light-permeable portion); after hitting an object, the light emitted by the light source chip 22 is reflected back to the proximity sensor 20; the reflected light enters the light-permeable portion of the housing 29 and reaches the first sensing area 251; according to the reflected light, the optical sensing chip 25 generates a sensing value for determining the distance or position of the object. In another embodiment, a lens may be arranged in the hole of the housing 29. In comparison with the conventional package structure 10 of the optical sensor shown in FIG. 1, the sunk-type package structure shown in FIG. 2 has the light source chip 22 and the optical sensing chip 25 disposed inside the cavity 211 of the substrate 21, whereby to decrease the thickness of the proximity sensor 20 and reduce the size of the proximity sensor 20. In the embodiment shown in FIG. 2, the light source chip 22 and the optical sensing chip 25 have an identical thickness of d2, and the thickness d2 is smaller than or equal to the first depth d1 of the cavity 211. However, the present invention is not limited by the embodiment. In another embodiment, the light source chip 22 and the optical sensing chip 25 may respectively have different thicknesses, and the thickness of the light source chip 22 or the optical sensing chip 25 may be larger than the first depth d1 of the cavity 211. In one embodiment, the first depth d1 is T/2±30 um, wherein T is the thickness of the substrate 21.

In FIG. 2, the substrate 21 has a solder mask 212 which is used to protect the wires (not shown in the drawing) on the substrate 21. The distance A between the edge of the solder mask 212 and the edge of the cavity 211 is greater than or equal to 200 um. The distance B between the edge of the cavity 211 and the edge of the light source chip 22 or the optical sensing chip 25 is greater than or equal to 75 um. The distance C between the edge of the cavity 211 and the proximity sensor 20 or the sunk-type package structure is greater than or equal to 100 um.

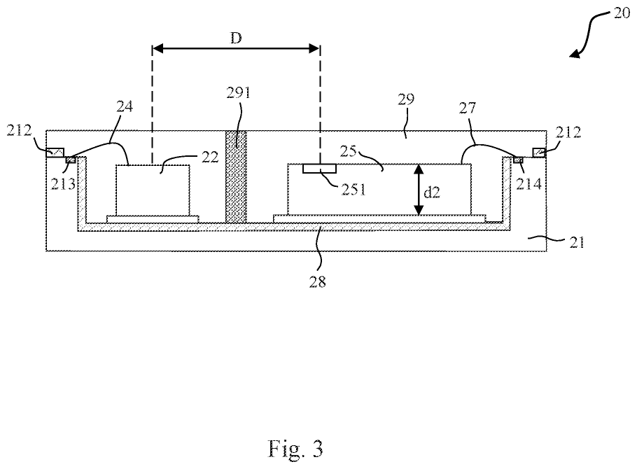

In FIG. 2, the light emitted from the lateral side of the light source chip 22 may enter the optical sensing chip 25 and affect the sensing result of the optical sensing chip 25. In one embodiment, for reducing the interference of the light source chip 22 to the optical sensing chip 25, the distance D between the central point of the light source chip 22 and the central point of the first sensing area 251 is greater than or equal to 0.8 mm. FIG. 3 schematically shows another embodiment of the housing 29. The difference between the proximity sensors 20 in FIG. 2 and FIG. 3 is that the housing 29 in FIG. 3 has an opaque shield wall 291 arranged between the light source chip 22 and the optical sensing chip 25 for shielding the light emitted from the lateral side of the light source chip 22 lest the light source chip 22 interfere with the optical sensing chip 25. In the embodiment shown in FIG. 3, because of the existence of the shield wall 291, the distance D between the central point of the light source chip 22 and the central point of the first sensing area 251 may be smaller than 0.8 mm.

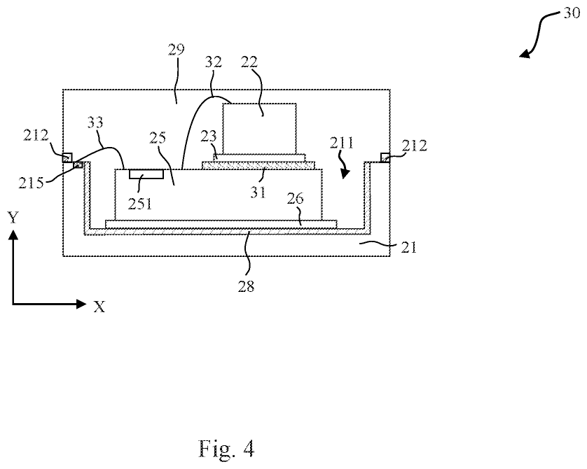

FIG. 4 schematically shows a second embodiment of a proximity sensor applying the sunk-type package structure of the present invention. Similar to the proximity sensor 20 in FIG. 2, the proximity sensor 30 in FIG. 4 also comprises a substrate 21, a light source chip 22, an optical sensing chip 25, a metal film 28 and a housing 29. The differences between the proximity 20 and the proximity sensor 30 are that the light source chip 22 is stacked over the optical sensing chip 25 and that the proximity sensor 30 has a grounding pad 31. In FIG. 4, the grounding pad 31 is disposed on the optical sensing chip 25 and near the first sensing area 251 of the optical sensing chip 25. The light source chip 22 is fixed to the grounding pad 31 with an adhesive 23 and electrically connected with the optical sensing chip 25 through a wire 32. The optical sensing chip 25 is fixed to the metal film 28 on the bottom of the cavity 211 with an adhesive 26 and electrically connected with a conduction pad 215 of the substrate 21 through a wire 33. In comparison with the conventional package structure 10 of the optical sensor, the thickness of the proximity sensor 30 is slightly less than or equal to the thickness of the package structure 10 in the Y direction, and the length of the proximity sensor 30 is reduced and smaller than the package structure 10 in the X direction. Therefore, the proximity sensor 30 is smaller in size. In the case that the housing 29 is fully transparent, because the light source chip 22 is stacked over the optical sensing chip 25, the optical crosstalk interference from the light source chip 22 to the optical sensing chip 25 is smaller in comparison with the architectures in FIG. 1 and FIG. 2.

In one embodiment, the distance D between the central point of the light source chip 22 and the central point of the first sensing area 251 in FIG. 4 is greater than or equal to 0.8 mm so as to reduce the interference of the light source chip 22 to the first sensing area 251 of the optical sensing chip 25. In another embodiment, the housing 29 in FIG. 4 has an opaque shield wall 291 (not shown in the drawing) arranged between the light source chip 22 and the optical sensing chip 25 for preventing the light source chip 22 from interfering with the first sensing area 251 of the optical sensing chip 25.

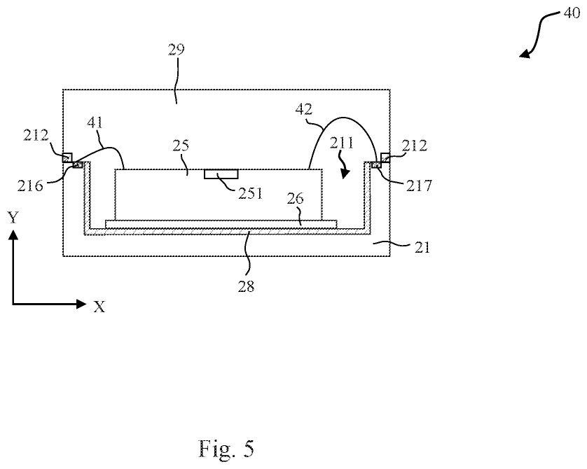

FIG. 5 schematically shows a first embodiment of an ambient light sensor applying the sunk-type package structure of the present invention. The ambient light sensor 40 shown in FIG. 5 comprises a substrate 21, an optical sensing chip 25, a metal film 28 and a housing 29. The substrate 21 has a solder mask 212 which is used to protect the wires (not shown in the drawing) on the substrate 21. The metal film 28 is formed on the surfaces of the inner walls and bottom of a cavity 211 of the substrate 21. The metal film 28 is used to reduce the electromagnetic interference (EMI) from the environment and the optical crosstalk interference from the substrate 21. The optical sensing chip 25 is disposed inside the cavity 211, fixed to the metal film 28 on the bottom of the cavity 211 with an adhesive 26 and electrically connected with conduction pads 216 and 217 of the substrate 21 through wires 41 and 42. The surface of the optical sensing chip 25 has a first sensing area 251 which configured to senses the light entering the ambient light sensor 40 to generate a sensing value for determining the intensity of the ambient light. The housing 29 covers the substrate 21 and the optical sensing chip 25. In the embodiment shown in FIG. 5, the housing 29 is made of a fully-transparent material. In other words, the complete housing 29 is a light-permeable portion allowing light to pass. In another embodiment, the housing 29 is made of an opaque material; a hole is formed on a region of the housing 29, which is above the first sensing area 251, to function as a light-permeable portion allowing external light to enter. In another embodiment, a lens may be arranged in the hole of the housing 29. In comparison with the conventional ambient light sensor 10, the sunk-type package structure shown in FIG. 5 has the optical sensing chip 25 disposed inside the cavity 211 of the substrate 21, whereby to decrease the thickness of the ambient light sensor 40 and reduce the size of the ambient light sensor 40.

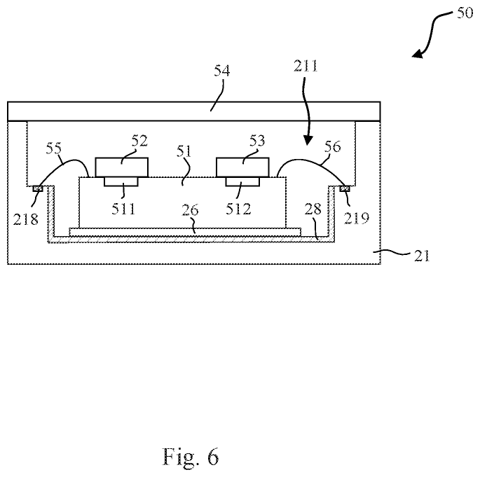

FIG. 6 schematically shows a second embodiment of an ambient light sensor applying the sunk-type package structure of the present invention. The ambient light sensor 50 shown in FIG. 6 comprises a substrate 21, an optical sensing chip 51, a metal film 28, a first polarization plate 52, a second polarization plate 53, and a quarter-wave plate 54. The metal film 28 is formed on the surfaces of the inner walls and bottom of a cavity 211 of the substrate 21. The metal film 28 is used to reduce the electromagnetic interference (EMI) from the environment and the optical crosstalk interference from the substrate 21. The optical sensing chip 51 is disposed inside the cavity 211, fixed to the metal film 28 on the bottom of the cavity 211 with an adhesive 26 and electrically connected with conduction pads 218 and 219 of the substrate 21 through wires 55 and 56. The optical sensing chip 51 has a first sensing area 511 and a second sensing area 512. The first sensing area 511 senses light to generate a first sensing value. The second sensing area 512 senses light to generate a second sensing value. The optical sensing chip 51 determines the intensity of the ambient light according to the first sensing value and the second sensing value. The first polarization plate 52 is disposed on the optical sensing chip 51 and covers the first sensing area 511. The second polarization plate 53 is disposed on the optical sensing chip 51 and covers the second sensing area 512. The first polarization plate 52 and the second polarization plate 53 respectively have a first polarization direction and a second polarization direction and both are used to filter light, wherein the first polarization direction is vertical to the second polarization direction. The quarter-wave plate 54 is disposed above the first polarization plate 52 and the second polarization plate 53 to function as the housing of the package structure. The quarter-wave plate 54 is a light-permeable element. Therefore, the quarter-wave plate 54 can be regarded as the light-permeable portion of the package housing. In comparison with the conventional ambient light sensor 10 in FIG. 1, the sunk-type package structure shown in FIG. 6 has the optical sensing chip 51 disposed inside the cavity 211 of the substrate 21, whereby to decrease the thickness of the ambient light sensor 50 and reduce the size of the ambient light sensor 50.

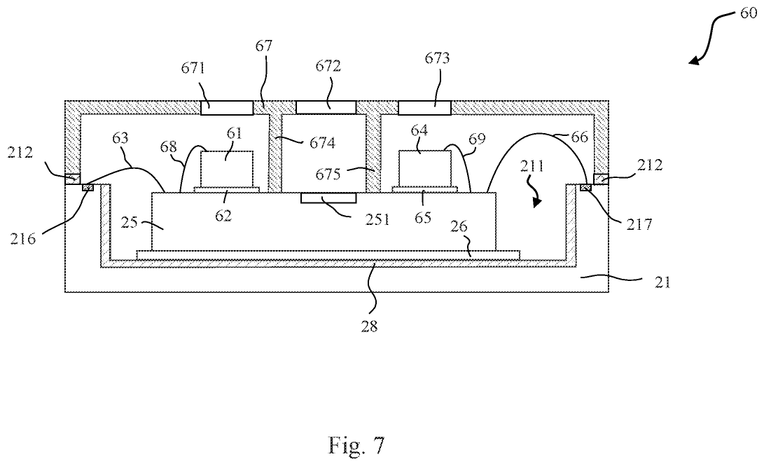

FIG. 7 schematically shows an embodiment of a heartbeat sensor applying the sunk-type package structure of the present invention. The heartbeat sensor 60 shown in FIG. 7 comprises a substrate 21, an optical sensing chip 25, a first light source chip 61, a second light source chip 64, a metal film 28, and a housing 67. The substrate 21 has a solder mask 212, which is used to protect the wires (not shown in the drawing) on the substrate 21. The metal film 28 is formed on the surfaces of the inner walls and bottom of a cavity 211 of the substrate 21. The metal film 28 is used to reduce the electromagnetic interference (EMI) from the environment and the optical crosstalk interference from the substrate 21. The optical sensing chip 25 is disposed inside the cavity 211, fixed to the metal film 28 on the bottom of the cavity 211 with an adhesive 26 and electrically connected with conduction pads 216 and 217 of the substrate 21 through wires 63 and 66. The first light source chip 61 is fixed to the optical sensing chip 25 with an adhesive 62 and electrically connected with the optical sensing chip 25 through a wire 68. The second light source chip 64 is fixed to the optical sensing chip 25 with an adhesive 65 and electrically connected with the optical sensing chip 25 through a wire 69. The first light source chip 61 and the second light source chip 64 respectively emit lights having different wavelengths. The housing 67 is made of an opaque package material. Light-permeable portions 671, 672, and 673 are formed on the housing 67 and respectively corresponding to the first light source chip 61, the first sensing area 251 of the optical sensing chip 25, and the second light source chip 64. In one embodiment, the light-permeable portions 671, 672 and 673 may be holes or lenses. A shield wall 674 of the housing 67 is disposed between the first sensing area 251 and the first light source chip 61, whereby to prevent the first light source chip 61 from interfering with the first sensing area 251. A shield wall 675 of the housing 67 is disposed between the first sensing area 251 and the second light source chip 64, whereby to prevent the second light source chip 64 from interfering with the first sensing area 251. Through the light-permeable portions 671 and 673, the lights, which are respectively emitted by the first light source chip 61 and the second light source chip 64, hit a human body. The light is reflected by the skin or blood vessels of the human body and then reaches the first sensing area 251 through the light-permeable portion 672. The first sensing area 251 senses the reflected light to generate a sensing value for determining the heartbeat value. In the sunk-type package structure shown in FIG. 7, the optical sensing chip 25 is disposed inside the cavity 211 of the substrate 21, and the first light source chip 61 and the second light source chip 64 are stacked over the optical sensing chip 25, whereby to decrease the thickness of the heartbeat sensor 60 and reduce the size of the heartbeat sensor 60.

In one embodiment, the housing 67 in FIG. 7 does not have the shield walls 674 and 675. In order to prevent the first light source chip 61 and the second light source chip 64 from interfering with the optical sensing chip 25, the distance between the central point of the first light source chip 61 and the central point of the first sensing area 251 is greater than or equal to 0.8 mm; the distance between the central point of the second light source chip 64 and the central point of the first sensing area 251 is also greater than or equal to 0.8 mm.

In the abovementioned embodiments, the substrate 21 may be but not limited to a coreless carrier plate.

In the abovementioned embodiments, the metal film 28 is formed inside the cavity 211. However, the metal film 28 may be canceled in other embodiments.

In the abovementioned embodiments, two proximity sensors, two ambient light sensors and a heartbeat sensor are used to exemplify the sunk-type package structure of the present invention. However, other optical sensors may also adopt the sunk-type package structure of the present invention.

The embodiments described above are only to exemplify the present invention but not to limit the scope of the present invention. Through making some modification or variation according to the technical contents disclosed in the specification and claims, any person having ordinary knowledge of the art should be able to generate equivalent embodiments without departing from the present invention. Further, the equivalent embodiments are to be included by the scope of the present invention.

What is claimed is:

1. A sunk-type package structure, comprising:
a substrate, having a cavity with a first depth;
an optical sensing chip, disposed inside the cavity and electrically connected with the substrate, wherein a surface of the optical sensing chip has a first sensing area for sensing light; and
a housing, covering the substrate and the optical sensing chip and having a light-permeable portion disposed above the first sensing area.

2. The sunk-type package structure according to claim 1, further comprising a metal film formed on inner walls and a bottom of the cavity, wherein the optical sensing chip is disposed on the metal film.

3. The sunk-type package structure according to claim 1, further comprising:
a grounding pad, disposed on the optical sensing chip and near the first sensing area; and
a light source chip, disposed on the grounding pad and configured to emit light, wherein the light source chip is electrically connected with the grounding pad.

4. The sunk-type package structure according to claim 3, wherein the housing includes a shield wall, which is disposed between the first sensing area and the light source chip.

5. The sunk-type package structure according to claim 3, wherein a distance between a central point of the light source chip and a central point of the first sensing area is greater than or equal to 0.8 mm.

6. The sunk-type package structure according to claim 2, further comprising:
a light source chip, disposed on the metal film and configured to emit light, wherein the housing includes a shield wall disposed between the optical sensing chip and the light source chip.

7. The sunk-type package structure according to claim 1, wherein the surface of the optical sensing chip further has a second sensing area for sensing light, and the sunk-type package structure further comprises:
a first polarization plate, covering the first sensing area and having a first polarization direction; and
a second polarization plate, covering the second sensing area and having a second polarization direction that is vertical to the first polarization direction;
wherein the light-permeable portion includes a quarter-wave plate disposed above the first polarization plate and the second polarization plate.

8. The sunk-type package structure according to claim 1, wherein a distance between an edge of a solder mask of the substrate and an edge of the cavity is greater than or equal to 200 um; a distance between the edge of the cavity and an edge of the optical sensing chip is greater than or equal to 75 um; a distance between the edge of the cavity and an edge of the sunk-type package structure is greater than or equal to 100 um.

9. The sunk-type package structure according to claim 1, wherein the substrate has a thickness of T, and the first depth is T/2±30 um.

10. The sunk-type package structure according to claim 1, wherein the substrate is a coreless carrier plate.

11. The sunk-type package structure according to claim 1, wherein a thickness of the optical sensing chip is smaller than or equal to the first depth.
